(19) United States
(12) Patent Application Publication
(10) Pub. No.: US 2005/0053956 A1
(43) Pub. Date: Mar. 10, 2005

(54) Detection of a predisposition for the development of coronary artery disease

(76) Inventors: Harry C. Dietz, Towson, MD (US); Dan E. Arking, Baltimore, MD (US)

Correspondence Address: Peter F. Corless, Esq.;Edwards & Angell, LLP, P.O. Box 55874, Boston, MA, 02205, US

(21) Appl. No.: 10/714,470

(22) Filed: Nov. 13, 2003

Related U.S. Application Data
(60) Provisional application No. 60/425,865, filed on Nov. 13, 2002.

(51) Int. Cl. C12Q 1/68; C12P 19/34

(52) U.S. Cl. 435/6; 435/91.2

(57) ABSTRACT

Methods and assays are disclosed for predicting a patient's predisposition for developing coronary artery disease or related vascular disorders. The methods comprise obtaining a biological sample from a patient and determining the presence or absence of the KL-VS allele which is linked with coronary artery disease. Detection of the allele is indicative of a predisposition or propensity to develop coronary artery disease. Kits for the detection of coronary artery disease are additionally provided.

This application claims priority from U.S. Provisional Application No. 60/425,865, filed Nov. 13, 2002, which is incorporated herein by reference.

### FIELD OF THE INVENTION

The invention relates to a specific genetic allele and predictive assay used in the presymptomatic identification of patients more probable to develop coronary artery disease and/or related vascular disorders than patients without the specific allele.

### BACKGROUND OF THE INVENTION

Coronary artery disease (CAD) usually results from atherosclerosis, a condition that occurs when arteries become narrow and hardened due to cholesterol plaque build-up. Further narrowing of the arteries may result from thrombi (blood clots) that form on the surfaces of plaques. Angina (chest pain) or dyspnea (shortness of breath) may be present.

Myocardial infarction (heart attack) can be a serious result of CAD, occurring when a blocked coronary artery causes death to a portion of the myocardium (heart muscle). Cardiac arrest may also result from CAD; 90% of sudden deaths occur in patients with two or more major arteries narrowed by atherosclerosis.

Statistics show CAD to be the leading cause of death among both men and women in the United States and in Europe. For example, approximately 12,800,000 Americans suffer from CAD and nearly 500,000 Americans die from heart attacks caused by CAD. Over 12 million Americans have a history of myocardial infarction or angina or both.

Similarly, two million Europeans die from CAD each year. Death rates from CAD are higher in Northern, Central and Eastern Europe and lower in Southern and Western Europe. For example, the death rate for men aged 35-74 living in Russia is eight times higher than in France and for women it is 12 times higher in Russia than in France Overall, an estimated 21% of European men and 22% of European women die from CAD.

Coronary artery disease is a multifactorial disease that results in the deposition of atheromatous plaque and progressive luminal narrowing of the arteries that supply the heart muscle. This plaque consists of a mixture of inflammatory and immune cells, fibrous tissue, and fatty material such as low-density lipids (LDL) and modifications thereof, and α-lipoprotein. The luminal narrowing or blockage results in reduced ability to deliver oxygen and nutrients to the heart muscle, producing myocardial infarction, angina, unstable angina, and sudden ischemic death as heart failure. Though occlusion usually progresses slowly, blood supply may be cut off suddenly when a portion of the built-up arterial plaque breaks off and lodges somewhere in an artery to block it temporarily, or more usually, when thrombosis occurs within the arterial lumen. Depending on the volume of muscle distal to the blockage during such an attack, a portion of myocardial tissue may die, weakening the heart muscle and often leading to the death of the individual.

The causes and mechanisms of the atheromatous plaque buildup are not completely understood, though many theories exist. One theory on the pathogenesis of atherosclerosis involves the following stages: 1) endothelial cell dysfunction and/or injury, 2) monocyte recruitment and macrophage formation, 3) lipid deposition and modification, 4) vascular smooth muscle cell proliferation, and 5) synthesis of extracellular matrix. According to this theory, the initiation of atherosclerosis is potentially due to a form of injury, possibly from mechanical stress or from chemical stress. How the body responds to this injury then defines whether or not, and how rapidly, the injury deteriorates into an atherosclerotic lesion. This in turn can result in arterial luminal narrowing and damage to the heart tissue which depends on the blood flow of oxygen and nutrients.

Controllable risk factors for CAD include high blood cholesterol, hypertension, smoking, diabetes, obesity, lack of physical activity, and stress. Uncontrollable risk factors for CAD include gender, family history, race, and genetics.

Tests used to detect and diagnose CAD include physical exam, blood cholesterol tests, blood pressure measurements, electrocardiogram, stress test, chest x-ray, coronary angiography, echo cardiogram, cardiac magnetic resonance imaging, fast/multi-slice CT, and nuclear medicine imaging.

Though recent improvements in cardiovascular care have improved the life expectancy of coronary artery disease patients, this has been primarily from improvements in lowering lipid levels, limitation of damage after it has occurred, surgical restoration of blood supply, the suppression of abnormal heart rhythms and prevention of re-infarction. Little improvement has occurred, however, in early prevention of the disease by early diagnosis.

A key problem in treating coronary artery disease is proper diagnosis. Often the first sign of the disease is sudden death due to myocardial ischemia or myocardial infarction. Approximately half of all individuals who die of coronary artery disease die suddenly. Furthermore, for 40-60% of the patients who are eventually diagnosed as having coronary artery disease, myocardial infarction is the first presentation of disease. Unfortunately, approximately 40% of those initial events go unnoticed by the patient. For various reasons, the perception of symptoms by the patient does not correlate well with the total burden of coronary artery disease (Anderson & Kin, Am. Heart J. 123(5);1312-23 (1992)).

While the causes of atherosclerosis remain unknown, the proper diagnosis of susceptibility may provide patients sufficient time to reduce their risk of developing coronary artery disease. One method to reduce the risk of coronary artery disease is through alteration of patient lifestyle such as smoking cessation, exercise, weight loss, and stress reduction. Other methods include pharmaceutical intervention to treat hypertension, hypercholesterolemia, and diabetes, as well as the use of aspirin. Finally, genetic therapy promises to treat those rare genetic traits that result in a family history of cardiovascular disease (e.g., altered apolipoprotein metabolism).

The ability to identify high-risk individuals would allow physicians to focus preventive measures on those individuals who may gain the greatest benefit, and would provide strong incentives for those at risk to comply with such approaches.

Traditional methods for the diagnosis of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes, such as, for example, Alzheimer's disease. With the development of genetic testing, it is now possible to identify gene mutations which indicate a propensity to develop disease, even when the disease is of polygenic origin. The number of diseases that can be diagnosed by molecular biological methods continues to grow with increased understanding of the genetic basis of multifactorial disorders (see e.g., U.S. Pat. Nos. 4,582,788; 5,110,920; 4,801,531; 4,666,828; and 5,268,267).

Genetic testing (also called genetic screening, genotyping or molecular diagnostics) can be defined broadly as the testing of nucleic acid of a patient in an analytical capacity to determine if a patient contains mutations (or alleles or polymorphisms) that either cause a disease state or are “linked” to the mutation causing a disease state. Linkage refers to the phenomenon that DNA sequences which are close together in the genome have a tendency to be inherited together. Two sequences may also be linked because of some selective advantage of co-inheritance.

The early detection of a predisposition to genetic diseases presents the best opportunity for medical intervention. Early genetic diagnosis may improve the prognosis for a patient through supervision and early intervention before the clinically detectable disorder occurs. In cases where patients with similar symptoms are treated with variable success, sophisticated genetic testing can differentiate individual patients with subtle or undetectable differences and can lead to more suitable individual treatments. It is even possible that early intervention may one day involve methods such as gene therapy.

As such, it would be highly desirable to develop diagnostic assays which would detect a predisposition for the development of coronary artery disease.

### SUMMARY OF THE INVENTION

The present invention provides inter alia a novel method for the early detection of a predisposition or propensity to develop coronary artery disease and related vascular disorders. It also provides kits for the early detection of said predisposition.

Generally, the method of predicting increased risk for the development of coronary artery disease consists of detecting the presence of at least one copy of an allele known as KL-VS, which is a functional variant of the gene KLOTHO (MIM 604824). This allele is characterized by six single nucleotide polymorphisms that occur within an 800 bp region spanning exon 2 and flanking sequence. Of the three mutations in exon 2, one is silent, and two code for amino acid substitutions, F352V and C370S, that influence klotho metabolism. Having one or more copies of this allele indicates increased risk for coronary artery disease. Detecting the allele may be performed directly, by analyzing the DNA, or indirectly, by analyzing the RNA or protein products of the DNA.

In another embodiment, the invention can be described as the following: isolating a nucleic acid sample from the patient, identifying one or more of the KL-VS alleles present in the sample, and comparing to a control sample. Identification of the KL-VS allele from the patient to the control sample indicates the patient's predisposition to coronary artery disease.

Another embodiment of the invention is a kit for the detection of the KL-VS allele that is predictive of coronary artery disease. The kit generally includes at least one oligonucleotide complementary to a DNA sequence in the KLOTHO gene. The kit may also include a DNA sampling means, a DNA purification means, and PCR reagents. Further, the oligonucleotide may contain a detectable label. Preferred kits in general comprise means for analyzing DNA of a patient to detect the presence of the KL-VS allele.

In another aspect, the invention includes methods for treating a patient suffering or susceptible to coronary artery disease. Such methods include identifying and selecting a patient that is need of treatment, particularly a patient that has a KL-VS allele as may be determined by techniques disclosed herein. The selected patient is then treated for coronary artery disease. For example an effective amount and regime of a therapeutic agent may be administered to the selected patient, such as a nitrate (e.g. nitroglycerine as may be suitably administered sublingually, or an amyl nitrate, isosorbide dinitrate or pentaerythritol tetranitrate); lifestyle change such as to eliminate or reduce smoking, weight loss, increase or aerobic exercise and the like; or surgical intervention such as coronary arterial bypass surgery.

Other embodiments and advantages of the invention are set forth in part in the description which follows, and will be obvious from this description, or may be learned from the practice of the invention.

### BRIEF DESCRIPTION OF THE FIGURES

FIG. 1 is a bar graph illustration of the frequency of Occult CAD in the SIBS-I Sample Stratified by KLOTHO Genotype; unadjusted P value for trend=0.002; error bars represent 95% confidence intervals.

FIG. 2 is a bar graph illustration of the frequency of Occult CAD in the SIBS-I Sample Stratified by Hypertension and KLOTHO Genotype; heterozygous and homozygous KL-VS allele carriers were combined due to the small numbers in the latter group after stratification for hypertension; error bars represent 95% confidence intervals.

FIG. 3 is a bar graph illustration of the frequency of Occult CAD in the SIBS-I Sample Stratified by KLOTHO Genotype and Current Smoking Status; heterozygous and homozygous KL-VS allele carriers were combined due to the small numbers in the latter group after stratification for current smoking status; error bars represent 95% confidence intervals.

FIG. 4 is a graphic representation of the Relative Odds of Occult CAD Conferred by the KL-VS Allele with Increasing HDL-C Levels; closed squares represent SIBS-I normotensive smokers; closed triangles represent SIBS-II normotensives.

FIG. 5 represents the amino acid sequences of Secreted (SEQ ID No.: 1) and Membrane-Bound (SEQ ID NO.: 2) forms of KLOTHO.

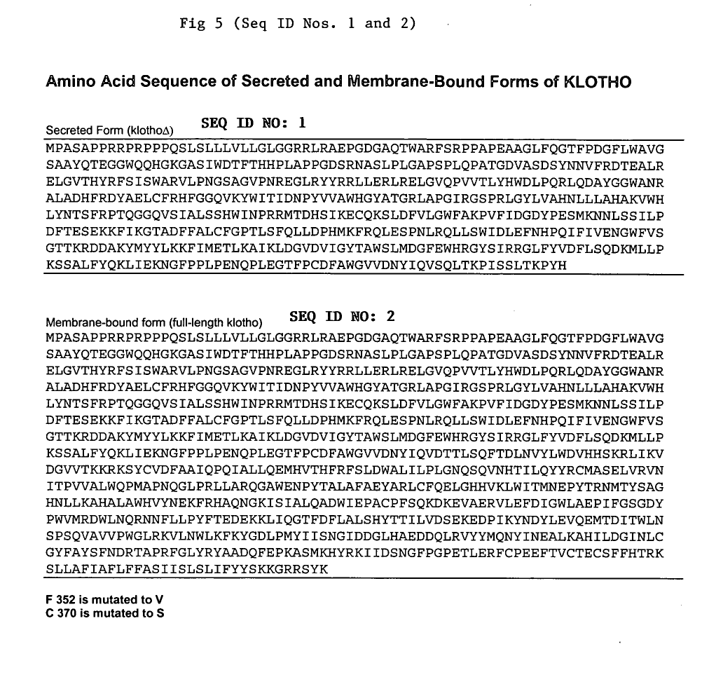

FIG. 6 represents the amino acid sequences of Secreted (SEQ ID No.: 3) and Membrane-Bound (SEQ ID NO.: 4) forms of KL-VS (KLOTHO with allelic variations with amino acid substitutions at positions 352, F→V and 370, C→S).

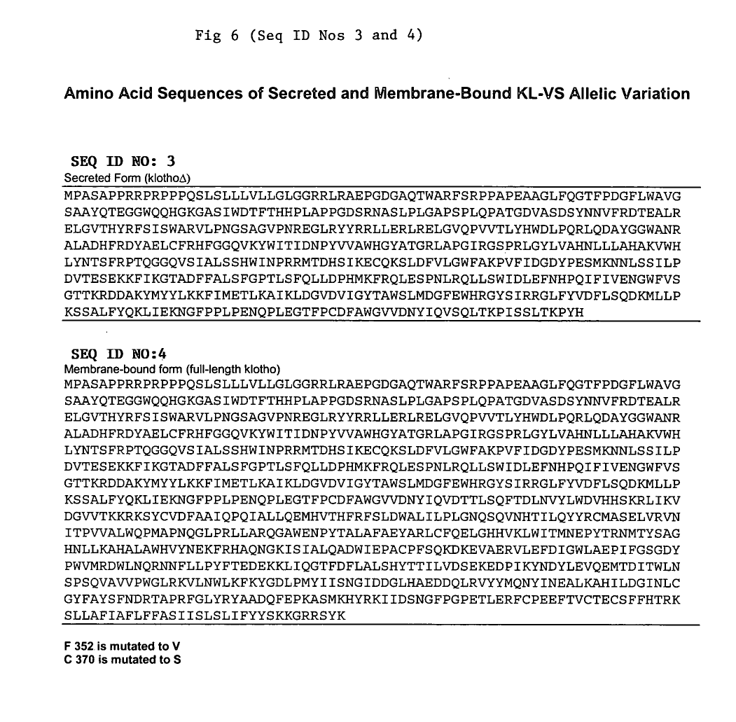

FIG. 7 represents the nucleotide sequence for the PCR sense primer (SEQ ID NO.: 5).

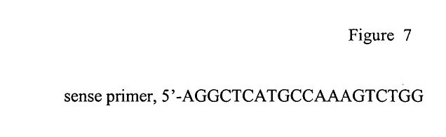

FIG. 8 represents the nucleotide sequence for the PCR anti-sense primer (SEQ ID NO.: 6).

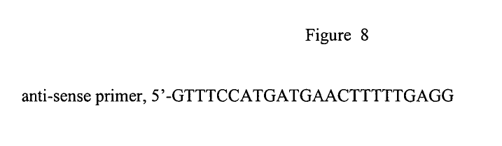

### DETAILED DESCRIPTION OF THE INVENTION

As embodied and broadly described herein, the present invention is directed to methods for predicting a patient's propensity toward developing coronary artery disease and to predictive and/or diagnostic kits, oligonucleotide probes and other reagents that can be used with these methods.

As used herein, the phrase coronary artery disease refers to disorders and conditions generally recognized by those skilled in the art as related to the deposition of atheroma in the large- and medium-sized arteries serving the heart. Thus, coronary artery disease means clinical syndromes (including, but not limited to, angina, myocardial infarction, unstable angina, and sudden ischemic death) which are based on the pathology of coronary artery atheroma.

The term marker is meant to describe regions of the DNA that vary between individuals. The different sequence variants at a given marker are called alleles or polymorphisms. Different alleles could have a single base change, including a substitution, insertion or deletion, or could have a change that affects multiple bases, including substitutions, insertions, deletions, repeats, inversions and combinations thereof. Alleles can be directly detected in the DNA of an individual, or indirectly detected in the RNA or protein. In the present invention, the KL-VS allele is characterized by six single nucleotide polymorphisms that occur within an 800 bp region spanning exon 2 and flanking sequence. Of the three mutations in exon 2, one is silent, and two code for amino acid substitutions, F352V and C370S, that influence klotho metabolism.

Klotho is a member of the family 1 glycosidases, and is composed of two internal repeats, each of which exhibits 20-40% sequence identity to β-glucosidases across phylogeny, as well as mammalian lactase phlorizin hydrolase. However, an enzymatic substrate for klotho has not been identified. Alternative RNA splicing yields both membrane-bound and secreted forms of klotho. Additionally, Klotho mRNA expression was not detectable in many organs in which severe pathology occurs in klotho-deficient mice. These observations and parabiosis experiments have led to the hypothesis that klotho acts as a humoral factor.

As used herein, the process of detecting alleles is variously described as genotyping, determining or identifying an allele or polymorphism, or any similar phrase. The allele actually detected might be a disease-causing mutation, or a mutation that is linked to a disease-causing mutation.

By propensity or predisposition or susceptibility to disease what is meant is that certain alleles are hereby discovered to be associated with a given disease state. They are thus over represented in individuals with disease as compared with healthy individuals. Therefore, the presence of such alleles indicates that an individual is at risk for the disease. In the present invention, the presence of the KL-VS allele indicates that an individual is at risk for developing coronary artery disease.

The invention is directed to a method of predicting the propensity or predisposition of a patient to develop coronary artery disease by genotyping the patient's DNA. The patient's genotype is compared with a control sample that contains DNA with the normal sequence of KLOTHO. The alleles in the sample may be in the form of genomic or cloned DNA sequences or may contain the end products appropriate for the assay format employed. For example, where the assay involves monoclonal detection of specific epitopes, the control samples might comprise the epitopes or proteins corresponding to the described allele, in this case, the KL-VS allele.

Techniques for determining the presence of particular markers may be nucleic acid techniques based on hybridization, size, or sequence, such as restriction fragment length polymorphism (RFLP) or nucleic acid sequencing.

These techniques may also comprise the step of amplifying the nucleic acid before analysis. Amplification techniques are known to those of skill in the art and include cloning, polymerase chain reaction (PCR), polymerase chain reaction of specific alleles (PASA), polymerase chain ligation, nested polymerase chain reaction, and the like. Amplification products may be assayed in a variety of ways, including size analysis, restriction digestion followed by size analysis, detecting specific tagged oligonucleotide primers in the reaction products, allele-specific oligonucleotide (ASO) hybridization, sequencing, and the like.

Alternatively, allele detection techniques may be protein based if a particular allele produces a protein with an amino acid variant. For example, epitopes specific for the amino acid variant can be detected with monoclonal antibodies.

Another embodiment of the invention is directed to diagnostic kits for detecting a propensity for coronary artery disease in a patient. The kits can be used presymptomatically or prenatally. The diagnostic kit may comprise one or more oligonucleotides capable of hybridizing to nucleic acid from the KLOTHO gene. A number of assay formats are useful for genotyping using the provided oligonucleotides. The most common formats involve nucleic acid binding, such as, for example, to filters, beads, or microtiter plates and the like. Techniques involved include dot blots, RNA blots, DNA blots, PCR, RFLP, and the like.

The oligonucleotides may be a variety of natural and synthetic compositions such as, for example, synthetic oligonucleotides, restriction fragments, cDNAs, synthetic PNAs (protein nucleic acids), and the like. The assay may also employ labeled oligonucleotides to allow ease of identification in the assays. Examples of labels which may be employed include radiolabels, enzymes, florescent compounds, streptavidin, avidin, biotin, magnetic moieties, metal binding moieties, antigen or antibody moieties, and the like.

The kit may also include DNA sampling means such as the AmpliCard.TM. (University of Sheffield, Sheffield, England S10 2JF), also described in Tarlow J W, et al. Journal of Investigative Dermatology 1994: 103: 387-389, incorporated by reference herein, or otherwise the Guthrie card. Other suitable DNA sampling means include DNA purification means and PCR reagents, such as 10× reaction buffers, thermostable polymerase, and/or dNTPs.

Other methods of detecting specific alleles and genotyping are described in U.S. Pat. Nos. 6,162,604; 5,945,289; 6,531,588; 6,479,242 and U.S. Patent Applications 20030096277; 20030186279; 20030143537; and 20030157517, all hereby incorporated by reference in their entirety.

All documents mentioned herein are incorporated herein by reference. The following example illustrates embodiments of the invention, but should not be viewed as limiting the scope of the invention.

Exemplification:

To determine whether KL-VS allele status influences risk for atherosclerotic CAD in humans and is related to known modifiable risk factors, cross-sectional association studies were employed in two independent samples of apparently healthy siblings of hospitalized index cases with documented early-onset (<60 years) CAD. Subjects who appeared healthy were chosen in order to enrich for factors which predispose to atherosclerosis as opposed to factors which may trigger cardiac events.

Materials and Methods of the Example

Study Population

This was a study of risk factors and their relationship to occult and incident CAD events in asymptomatic, apparently healthy 30-59 year old siblings (SIBS) of individuals hospitalized with documented CAD before 60 years of age. SIBS were asked to participate in a comprehensive cardiovascular screening protocol and were eligible if they were <60 years of age and had experienced no clinically manifest CAD. Two separate samples were recruited using the same methods and screened identically. The first sample (SIBS-I) was recruited from 1983 to 1996 from three Baltimore hospitals. The second sample (SIBS-II) was composed exclusively of African Americans and was recruited from 1998 to 2001 from 10 Baltimore hospitals. Recruitment methods are described in detail in a prior publication (Becker et al. 1998).

SIBS Risk Factor Assessment

A cardiovascular history and physical examination was performed and blood was obtained for measurement of fasting levels of plasma total cholesterol (TC), high density lipoprotein cholesterol (HDL-C), and triglycerides (TG) in the Johns Hopkins Chemistry laboratory which is CDC-standardized. Low density lipoprotein cholesterol (LDL-C) was estimated using the Friedewald formula for persons with TG levels <400 mg/dl (Friedewald et al. 1972). For adjusted analysis (see below), individuals with TG levels >400 mg/dl were assigned an average LDL-C for their race and sex group (n=29). Blood pressure was measured at standard intervals according to American Heart Association Guidelines (Association 1990) during an eight-hour screening day. Three readings were averaged to characterize blood pressure for all subjects. Hypertension was defined as an average blood pressure ≧140/90 mm Hg or the current use of antihypertensive medication. Current smoking status was defined by self-reported smoking of any cigarettes within the past month and validated by expired carbon monoxide levels >8 ppm (Morabia et al. 2001). Body-mass index was measured as weight in kilograms divided by the square of the height in meters.

SIBS Exercise Thallium Scintigraphy

All SIBS underwent a maximal symptom-limited graded exercise treadmill test (ETT) using a modified Bruce protocol (Blumenthal et al. 1996). In men, a positive stress test was defined as horizontal or downsloping ST-segment depression of ≧1 mm over baseline at 0.06 seconds after the J-point in ≧3 consecutive beats during the stress test or during the first 3 minutes of recovery. In women an abnormal response was defined as ≧2 mm flat or downsloping ST-segment depression over baseline in leads II, II, or aVF or ≧1.5 mm ST-segment depression in any other lead.

Thallium-201 scintigraphy was performed in conjunction with the ETT as previously described (Blumenthal et al. 1996). Briefly, one minute before the end of exercise, 3-4 mCi thallium-201 was injected intravenously, and tomographic imaging was begun 5-10 minutes later. After four hours, delayed imaging was performed. Images were reconstructed by filtered back projection using a ramp filter after prefiltering of the projection images with a 2-dimensional Fourier (Weiner) filter and correction for translational motion. Image interpretation was performed visually by an experienced nuclear cardiologist (LCB) without knowledge of the subject's identity, genotype, or risk factor results.

Occult CAD was defined as an abnormal thallium tomogram test represented by a segmental perfusion defect on the immediate post-exercise images in ≧2 contiguous tomographic slices and 2 image orientations accompanied by definite improvement or normalization on the delayed images. Using receiver operating curve analysis, the visual interpretation using these criteria provides a sensitivity for coronary disease detection of 95% and a false-positive rate of <10% (Fintel et al. 1989). In SIBS the presence of perfusion defects is strongly associated with incident premature CAD in the SIB population (Blumenthal et al. 1996).

KL-VS Screening

After DNA was extracted from whole blood buffy coat preparations or Guthrie cards according to standard protocols, sample DNA was amplified using PCR (sense primer, 5′-AGGCTCATGCCAAAGTCTGG; anti-sense primer, 5′-GTTTCCATGATGAACTTTTTGAGG) with AmpliTaq Gold (Perkin Elmer) with supplied buffer under the following conditions: 95° C. 10 min, followed by 35 cycles of 94° C. 30s, 60° C. 30s, 72° C. 30s, followed by a 10 min 72° C. final extension. PCR products were then digested with Mae III (Roche) at 55° C. for 16 hrs, and electrophoretically separated on a 1.6% agarose gel. The KL-VS allele is characterized by diagnostic Mae III restriction fragments of 265 and 185 basepairs.

Statistical Analysis

Descriptive statistics for each risk variable included means and standard deviations (continuous variables) or frequencies (qualitative variables) among occult CAD cases and control siblings separately. Unadjusted univariate comparisons between groups were performed via Shapiro-Wilk tests or the χ² statistic in SAS version 8.2 (SAS Institute Inc., Cary, N.C.; 2001). Adjusted analyses for KL-VS allele status and interactive effects were performed via general linear modeling using PROC GENMOD in SAS, employing a log-link function to reflect logistic regression, and using Generalized Estimating Equations (GEE) (Liang and Zeger 1993) to account for the familial intraclass correlations between siblings when obtaining parameter estimates and variances. Due to the limited number of KL-VS homozygotes, adjusted analyses including more than one interaction term assumed a dominant genetic model comparing KL-VS carriers to non-carriers. The influence of modifiable risk factors on the risk conferred by KL-VS allele status was evaluated via the inclusion of interaction terms and the significance was assessed via likelihood ratio testing. Odds ratios and confidence intervals were obtained via GEE methods.

### EXAMPLE

The characteristics of the study population shown in Table 1 are stratified by the independent sibling samples (SIBS-I and SIBS-II) and by occult CAD status. SIBS-I (n=520) was predominantly Caucasian and SIBS-II (n=436) was exclusively African American. The samples were middle-aged and those with occult CAD were slightly but significantly older in both SIBS samples. Overall, the risk factors were generally more prevalent in persons with occult CAD and the expected differences in levels and prevalence that were observed between SIBS-I and SIBS-II likely reflects racial composition and socioeconomic factors (Becker et al. 1998; Clark 1999).

TABLE 1
Risk Factor and Demographic Characteristics Stratified by Sample and
The Presence or Absence of Occult Coronary Disease*

| | SIBS-I | | | SIBS-II | | |
|---|---|---|---|---|---|---|
| | Occult CAD | Occult CAD | | Occult CAD | Occult CAD | |
| Characteristic | Absent N = 423 | Present N = 97 | P | Absent N = 380 | Present N = 56 | P |
| Age (years) | 45.3 ± 7.1 | 48.7 ± 7.0 | .0001 | 47.0 ± 6.7 | 49.0 ± 6.3 | .0367 |
| HDL-C mg/dl | 51.4 ± 16.0 | 46.4 ± 14.9 | .0050 | 54.4 ± 16.9 | 53.8 ± 15.9 | .7939 |
| LDL-C mg/dl | 147.4 ± 42.7 | 162.3 ± 44.7 | .0021 | 129.1 ± 39.2 | 136.2 ± 39.4 | .2021 |
| TG mg/dl | 149.1 ± 123.8 | 194.3 ± 208.7 | .0426 | 123.5 ± 84.7 | 109.5 ± 57.9 | .1184 |
| Systolic BP mmHg | 130.6 ± 14.2 | 138.8 ± 14.5 | .0001 | 135.5 ± 16.6 | 139.8 ± 12.6 | .0236 |
| Diastolic BP mmHg | 83.3 ± 9.4 | 87.7 ± 9.0 | .0001 | 86.5 ± 10.5 | 88.0 ± 8.3 | .2347 |
| BMI | 27.6 ± 5.8 | 28.5 ± 3.8 | .0478 | 31.9 ± 6.9 | 29.7 ± 5.4 | .0064 |
| % Diabetes | 6.4 | ta 3.1 | .2116 | 15.0 | 14.3 | .8886 |
| % Hypertension | 39.6 | 54.6 | .0069 | 58.9 | 67.9 | .2037 |
| % Current Smokers | 31.1 | 28.9 | .6625 | 31.6 | 32.1 | .9325 |
| % African American | 16.0 | 13.4 | .5181 | 100 | 100 | — |
| % Male | 46.5 | 76.3 | .0001 | 30.3 | 64.3 | .0001 |

Plus-minus values are mean ± SD. P values reflect t-tests or χ² tests comparing occult CAD status groups within each sample.
*Occult CAD = positive exercise electrocardiogram and/or positive thallium scan.

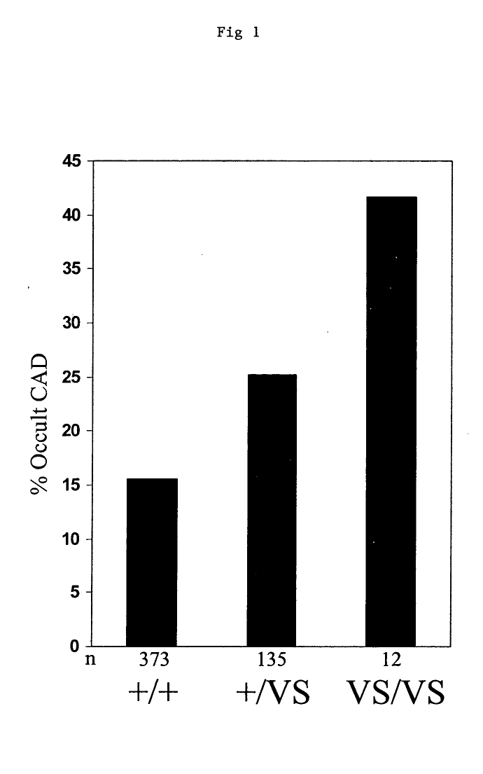

The previously identified functional variant of klotho, termed KL-VS, was known to be involved in human survival (Arking et al. 2002). To determine whether this allele also influences the risk for early-onset occult CAD. Both SIBS-I (0.153) and SIBS-II (0.164) have KL-VS allele frequencies similar to that seen in previous studies (Arking et al. 2002). FIG. 1 shows the frequency of occult CAD in SIBS-I stratified by KL-VS genotype. Univariate logistic regression analysis assuming an additive genetic model (on a logit scale) and incorporating a GEE adjustment for familial intraclass correlations indicates a relative odds of 1.90 (95% C.I. 1.21-2.98) for occult CAD conferred by the KL-VS allele (P<0.005). To test whether KL-VS allele status is an independent risk factor, a logistic regression model was constructed incorporating known CAD risk factors (LRM). Addition of KL-VS allele status significantly improved the model after adjusting for age, sex, race, body-mass index (BMI), hypertension (HTN or RX), HDL-C level, LDL-C level, triglyceride level (TG), diabetes, and current smoking status (P<0.019), indicating that KL-VS allele status is a novel risk factor for occult atherosclerosis in this sample.

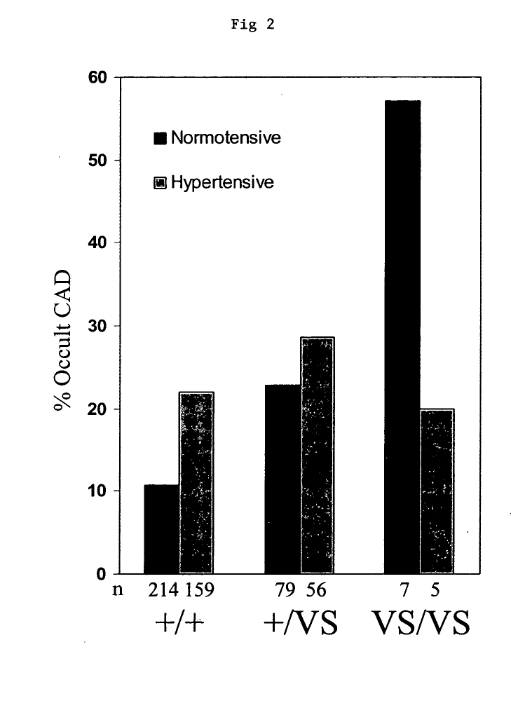

Experimental data in rats have demonstrated that klotho mRNA and protein levels are down-regulated under sustained circulatory stress (Nagai et al. 2000; Aizawa et al. 1998). It is therefore plausible that hypertensive conditions in humans could also lead to global downregulation of klotho. In this view, the presence of concomitant hypertension could modify the risk imposed by the KL-VS allele, which is believed to manifest relative loss of functional klotho. Incorporation of an interaction term into the LRM to test whether hypertension modifies the risk associated with the KL-VS allele (klotho*HTN or RX) significantly improved the model (P<0.022), and indicates that the detrimental effect of the KL-VS allele is more pronounced in normotensive individuals (FIG. 2 and Table 2).

TABLE 2
Multiple Logistic Regression Predicting Occult CAD
for SIBS-I and SIBS-II Samples*

| | SIBS-I | | | SIBS-II | | |
|---|---|---|---|---|---|---|
| Risk Factor | Beta | S.E. | P | Beta | S.E. | P |
| Intercept | −9.2071 | 1.7987 | 0.0001 | −3.0780 | 1.9622 | 0.12 |
| Age | 0.0971 | 0.0238 | 0.0001 | 0.0512 | 0.0302 | 0.09 |
| Sex | −1.4290 | 0.3709 | 0.0001 | −1.2623 | 0.3292 | 0.0001 |
| Race | −0.0690 | 0.3985 | 0.86 | — | — | — |
| LDL-C | 0.0062 | 0.0034 | 0.30 | 0.0077 | 0.0046 | 0.10 |
| HDL-C | 0.0190 | 0.0121 | 0.12 | 0.0040 | 0.0153 | 0.79 |
| TG | 0.0010 | 0.0007 | 0.17 | −0.0046 | 0.0030 | 0.13 |
| Diabetes | −1.0038 | 0.6767 | 0.14 | 0.5087 | 0.5145 | 0.32 |
| HTNorRX | 0.5211 | 0.3404 | 0.13 | 0.4938 | 0.4703 | 0.29 |
| BMI | 0.0478 | 0.0278 | 0.09 | −0.0585 | 0.0351 | 0.10 |
| Smoking | −0.4201 | 0.4039 | 0.30 | 0.0885 | 0.4470 | 0.84 |
| Klotho | 2.4170 | 1.0447 | 0.021 | 2.6908 | 1.3959 | 0.05 |
| Klotho*HTNor | −0.9834 | 0.5851 | 0.09 | −1.2558 | 0.7344 | 0.09 |
| RX | | | | | | |
| Klotho*Smok- | 1.4019 | 0.6477 | 0.030 | −0.0048 | 0.7282 | 0.99 |
| ing | | | | | | |
| Klotho*HDL-C | −.0383 | 0.0210 | 0.07 | −0.0341 | 0.0212 | 0.11 |
| Significance of | | | 0.0002 | | | 0.06 |
| overall klotho | | | | | | |
| effect** | | | | | | |

Klotho is incorporated under the assumption of a dominant model.
*GEE adjusted.
**Likelihood ratio tests between models including klotho and significant modifier terms and models without klotho terms.

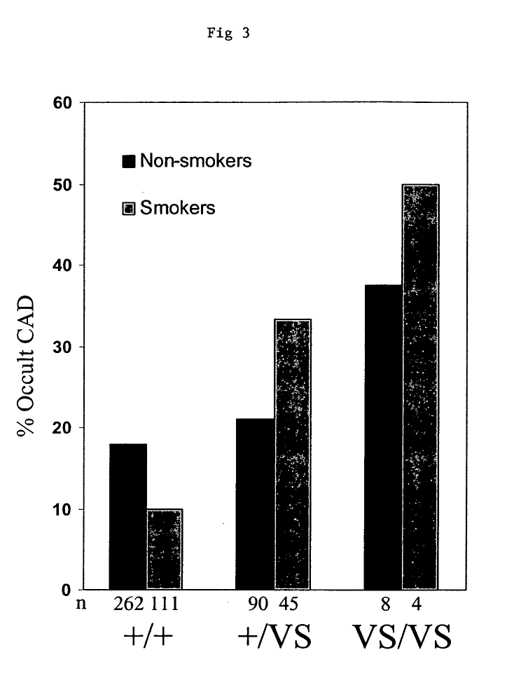

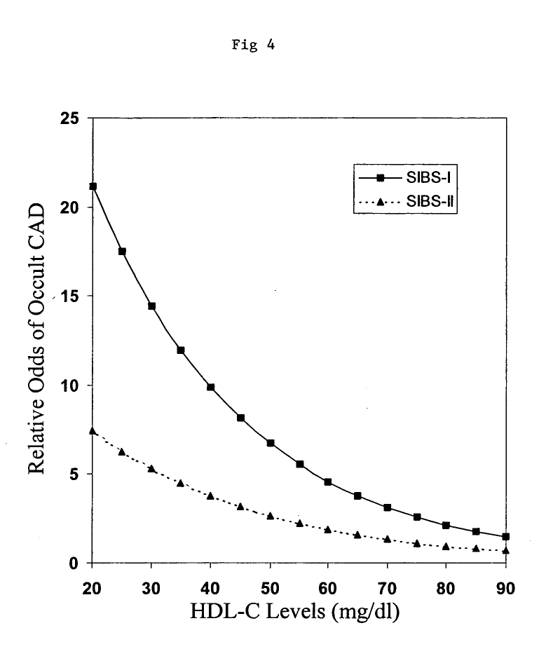

The possibility that additional modifiable risk factors influence the association between KL-VS allele status and occult atherosclerosis was analyzed by sequentially incorporating the risk factor interaction term that most significantly improved the LRM (P<0.05). The klotho term is incorporated under the assumption of a dominant model, due to insufficient homozygotes for analysis under the assumption of an additive model. Following this strategy, significant modifications to the risk associated with KL-VS allele status were observed for current smoking status (P<0.004) (FIG. 3 and Table 2) and HDL-C levels (P<0.022) (Table 2). The addition of KL-VS allele status, including the modifications of risk due to hypertension, smoking, and HDL-C levels, resulted in a highly significant increase in the overall fit of the LRM (P<0.0002), and indicate a relative odds of >9.8 (95% C.I. 3.27-29.6) for occult CAD among normotensive smokers with HDL-C levels ≦40 mg/dl who carry the KL-VS allele versus non-carriers (FIG. 4).

An additional sample (SIBS-II) was analyzed and recruited under the same criteria as the SIBS-I sample, where enrollment was limited to African-Americans (Table 1). Univariate logistic regression analysis did not indicate an increased risk for occult CAD for carriers of the KL-VS allele (data not shown). This result was somewhat predictable given the higher frequency of hypertension and the higher HDL levels in SIBSII. However, inclusion of KL-VS allele status in an LRM constructed to incorporate known risk factors and risk modifications due to hypertension and HDL-C level significantly improved the ability to predict individuals with occult CAD in SIBS-II (P<0.06), as previously observed in SIBS-I (P<0.0002) (Table 2). These results are particularly striking when one takes into account the remarkable similarity in the magnitude of effect attributable to the KL-VS allele and significant modifying risk factors in the two groups (Table 2, bolded values). Current smoking status had no effect on the risk for occult CAD conferred by the KL-VS allele in the SIBS-II population (P>0.99). In SIBSII, the relative odds for occult CAD is >3.8 (95% C.I. 1.29-10.9) among normotensive individuals with HDL-C levels ≦40 mg/dl who carry the KL-VS allele versus non-carriers (FIG. 4). These results confirm that KL-VS allele status is an important predictor of occult CAD in family members of individuals with early-onset and clinically manifest CAD. However, modification of the risk conferred by the KL-VS allele by known CAD risk factors may not be identical in all populations.

Family history of premature coronary artery disease is a major predictor for CAD in a first degree relative even after adjusting for known risk factors, suggesting the presence of as yet unidentified genetic contributors to this complex disease (Shea et al. 1984; Schildkraut et al. 1989). Previously, the KL-VS allele of the KLOTHO gene was identified as a functional variant that influences longevity, and demonstrated that the derived protein likely has reduced activity (Arking et al. 2002). Individuals who are homozygous for this allele exhibit a 2.6-fold reduction in survival to ≧65 years of age. Based in part on observations in klotho-deficient mice, a role for klotho in protecting the cardiovascular system has been proposed (Saito et al. 1998; Nagai et al. 2000). The present study now demonstrates that the KL-VS allele of KLOTHO confers risk for occult atherosclerosis in a high-risk sample composed of siblings of individuals with premature CAD. The effect of the KL-VS allele is evident after adjusting for known risk factors, including sex, indicating that it is a novel risk factor for both men and women. Modifiable risk factors, including hypertension, current smoking, and HDL-C level significantly modulated the risk associated with the KL-VS allele. To confirm these results, an additional independent sample was analyzed. A univariate klotho effect in this sample was not observed, due to the increased levels of HDL-C and prevalence of hypertension in the sample, factors which mitigate the contribution of the KL-VS allele to increased risk for occult CAD. However, in a LRM we did observe striking similarity in klotho and klotho*HDL/HTN modifier effects between the two samples.

The identification of known modifiable risk factors that influence the risk conferred by the KL-VS allele is of particular interest, as they may shed light on the mechanism by which klotho influences atherogenic CAD risk, and suggest productive strategies for therapeutic intervention. Hypertension and HDL-C levels significantly modified the risk associated with KL-VS allele status in both samples. Normotensive KL-VS carriers demonstrated a greater increase in risk for occult atherosclerosis than hypertensive carriers. Experimental data generated in rat models demonstrates that klotho levels are down-regulated under hypertensive conditions (Nagai et al. 2000; Aizawa et al. 1998), and specifically by angiotensin II (Mitani et al. 2002). Thus, the levels of klotho are likely to be reduced in hypertensive individuals, which could mask the detrimental effect of the KL-VS allele. This hypothesis also raises the intriguing question of whether the status of elevated angiotensin II as an independent risk factor for cardiovascular disease (Brunner 2001; Gavras and Gavras 2002) may be due, at least in part, to downregulation of klotho levels. Since a system for measuring serum levels of klotho has not yet been developed, this hypothesis remains to be tested. Alternatively, the modification of the risk conferred by the KL-VS allele due to hypertension could arise if hypertensive individuals who carry the KL-VS allele exhibit greatly increased coronary events, as only individuals who are apparently unaffected were included in this study. Similarly, a differential response to antihypertensive medication due to KL-VS genotype could contribute to this result. Prospective studies are required to address these hypotheses.

Klotho has been proposed to protect the cardiovascular system through endothelium derived NO production (Saito et al. 1998; Saito et al. 2000; Nagai et al. 2000; Fukino et al. 2002), suggesting that the KL-VS allele may increase the risk for occult CAD due, at least in part, to a decrease in NO production. Thus, in light of recent evidence that HDL-C activates endothelial nitric oxide synthase (eNOS) and increases NO expression (Yuhanna et al. 2001; Kuvin et al. 2002), it is particular intriguing that increased HDL-C levels specifically protect against the detrimental effect associated with the KL-VS allele. These data are consistent with a model that invokes opposing influences of klotho deficiency and elevated HDL-C levels on NO production and ultimate risk of CAD. This result raises the possibility that the use of drugs such as HMG CoA-reductase inhibitors (statins) or niacin to increase HDL-C levels may prove to be particularly effective in preventing CAD in KL-VS carriers, and that intervention might be productive even in cases with normal HDL-C levels.

Electronic-Database Information

Accession numbers and URLs for data presented herein are as follows:
Online Mendelian Inheritance in Man (OMIM), http://www.ncbi.nlm.nih.gov/Omim (for KLOTHO [MIM 604824])

### REFERENCES

Ahijevych K (1999) Nicotine metabolism variability and nicotine addiction. Nicotine Tob Res 1:S59-62; discussion S69-70.
Aizawa H, Saito Y, Nakamura T, Inoue M, Imanari T, Ohyama Y, Matsumura Y, Masuda H, Oba S, Mise N, Kimura K, Hasegawa A, Kurabayashi M, Kuro-o M, Nabeshima Y, Nagai R (1998) Downregulation of the Klotho gene in the kidney under sustained circulatory stress in rats. Biochem Biophys Res Commun 249:865-71.
Arking D E, Krebsova A, Macek M, Sr., Macek M, Jr., Arking A, Mian I S, Fried L, Hamosh A, Dey S, McIntosh I, Dietz H C (2002) Association of human aging with a functional variant of klotho. Proc Natl Acad Sci USA 99:856-61.
Association A H (1990) Human Blood Pressure Determination by Sphygmomanometry. American Heart Association, Dallas, pp 2460-2467
Becker D M, Yook R M, Moy T F, Blumenthal R S, Becker L C (1998) Markedly high prevalence of coronary risk factors in apparently healthy African-American and white siblings of persons with premature coronary heart disease. Am J Cardiol 82:1046-51.
Blumenthal R S, Becker D M, Moy T F, Coresh J, Wilder L B, Becker L C (1996) Exercise thallium tomography predicts future clinically manifest coronary heart disease in a high-risk asymptomatic population. Circulation 93:915-23.
Brunner H R (2001) Experimental and clinical evidence that angiotensin II is an independent risk factor for cardiovascular disease. Am J Cardiol 87:3C-9C.
Clark L T (1999) Primary prevention of cardiovascular disease in high-risk patients: physiologic and demographic risk factor differences between African American and white American populations. Am J Med 107:22S-24S.
Denke M A, Adams-Huet B, Nguyen A T (2000) Individual cholesterol variation in response to a margarine- or butter-based diet: A study in families. Jama 284:2740-7.
Fintel D J, Links J M, Brinker J A, Frank T L, Parker M, Becker L C (1989) Improved diagnostic performance of exercise thallium-201 single photon emission computed tomography over planar imaging in the diagnosis of coronary artery disease: a receiver operating characteristic analysis. J Am Coll Cardiol 13:600-12.
Friedewald W T, Levy R I, Fredrickson D S (1972) Estimation of the concentration of low-density lipoprotein cholesterol in plasma, without use of the preparative ultracentrifuge. Clin Chem 18:499-502.
Fukino K, Suzuki T, Saito Y, Shindo T, Amaki T, Kurabayashi M, Nagai R (2002) Regulation of angiogenesis by the aging suppressor gene klotho. Biochem Biophys Res Commun 293:332-7.
Gavras I, Gavras H (2002) Angiotensin II as a cardiovascular risk factor. J Hum Hypertens 16 Suppl 2:S2-6.
Hines L M, Stampfer M J, Ma J, Gaziano J M, Ridker P M, Hankinson S E, Sacks F, Rimm E B, Hunter D J (2001) Genetic variation in alcohol dehydrogenase and the beneficial effect of moderate alcohol consumption on myocardial infarction. N Engl J Med 344:549-55.
Kuro-o M, Matsumura Y, Aizawa H, Kawaguchi H, Suga T, Utsugi T, Ohyama Y, Kurabayashi M, Kaname T, Kume E, Iwasaki H, Iida A, Shiraki-Iida T, Nishikawa S, Nagai R, Nabeshima Y I (1997) Mutation of the mouse klotho gene leads to a syndrome resembling ageing. Nature 390:45-51.
Kuvin J T, Ramet M E, Patel A R, Pandian N G, Mendelsohn M E, Karas R H (2002) A novel mechanism for the beneficial vascular effects of high-density lipoprotein cholesterol: enhanced vasorelaxation and increased endothelial nitric oxide synthase expression. Am Heart J 144:165-72.
Liang K Y, Zeger S L (1993) Regression analysis for correlated data. Annu Rev Public Health 14:43-68
McCombs R J, Marcadis D E, Ellis J, Weinberg R B (1994) Attenuated hypercholesterolemic response to a high-cholesterol diet in subjects heterozygous for the apolipoprotein A-IV-2 allele. N Engl J Med 331:706-10.
Mitani H, Ishizaka N, Aizawa T, Ohno M, Usui S, Suzuki T, Amaki T, Mori I, Nakamura Y, Sato M, Nangaku M, Hirata Y, Nagai R (2002) In vivo klotho gene transfer ameliorates angiotensin II-induced renal damage. Hypertension 39:838-43.
Morabia A, Bernstein M S, Curtin F, Berode M (2001) Validation of self-reported smoking status by simultaneous measurement of carbon monoxide and salivary thiocyanate. Prev Med 32:82-8.
Nagai R, Saito Y, Ohyama Y, Aizawa H, Suga T, Nakamura T, Kurabayashi M, Kuroo M (2000) Endothelial dysfunction in the klotho mouse and downregulation of klotho gene expression in various animal models of vascular and metabolic diseases. Cell Mol Life Sci 57:738-46.
Ordovas J M (1999) The genetics of serum lipid responsiveness to dietary interventions. Proc Nutr Soc 58:171-87.
Saito Y, Nakamura T, Ohyama Y, Suzuki T, Iida A, Shiraki-Iida T, Kuro-o M, Nabeshima Y, Kurabayashi M, Nagai R (2000) In vivo klotho gene delivery protects against endothelial dysfunction in multiple risk factor syndrome. Biochem Biophys Res Commun 276:767-72.
Saito Y, Yamagishi T, Nakamura T, Ohyama Y, Aizawa H, Suga T, Matsumura Y, Masuda H, Kurabayashi M, Kuro-o M, Nabeshima Y, Nagai R (1998) Klotho protein protects against endothelial dysfunction. Biochem Biophys Res Commun 248:324-9.
Schildkraut J M, Myers R H, Cupples L A, Kiely D K, Kannel W B (1989) Coronary risk associated with age and sex of parental heart disease in the Framingham Study. Am J Cardiol 64:555-9.
Shea S, Ottman R, Gabrieli C, Stein Z, Nichols A (1984) Family history as an independent risk factor for coronary artery disease. J Am Coll Cardiol 4:793-801.
Shiraki-Iida T, Iida A, Nabeshima Y, Anazawa H, Nishikawa S, Noda M, Kuro-o M (2000) Improvement of multiple pathophysiological phenotypes of klotho (kl/kl) mice by adenovirus-mediated expression of the klotho gene. J Gene Med 2:233-42.
Talmud P J, Bujac S R, Hall S, Miller G J, Humphries S E (2000) Substitution of asparagine for aspartic acid at residue 9 (D9N) of lipoprotein lipase markedly augments risk of ischaemic heart disease in male smokers. Atherosclerosis 149:75-81.
Woods M N, Harris K J, Ahluwalia J S, Schmelzle K H, Mayo M S (2001) Smoking in urban African Americans: behaviors, gender differences, and motivation to quit. Ethn Dis 11:532-9.
Yuhanna I S, Zhu Y, Cox B E, Hahner L D, Osborne-Lawrence S, Lu P, Marcel Y L, Anderson R G, Mendelsohn M E, Hobbs H H, Shaul P W (2001) High-density lipoprotein binding to scavenger receptor-BI activates endothelial nitric oxide synthase. Nat Med 7:853-7.

General methods in molecular biology: Standard molecular biology techniques known in the art and not specifically described were generally followed as in Sambrook et al., Molecular Cloning: A Laboratory Manual, Cold Spring Harbor Laboratory Press, New York (1989); in Ausubel et al., Current Protocols in Molecular Biology, John Wiley and Sons, Baltimore, Md. (1989); in Perbal, A Practical Guide to Molecular Cloning, John Wiley & Sons, New York (1988); in Watson et al., Recombinant DNA, Scientific American Books, *New York; in Birren et al. (eds), Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al. 1996, Blood 87:3822.). All publications, patents and patent applications (including U.S. Provisional Patent application 60/337,315, entitled “Klotho and Aging”, filed 12-6-02) disclosed herein are incorporated by reference in their entirety into this application.

The foregoing description of the invention is merely illustrative thereof, and it is understood that variations and modifications can be effected without departing from the scope of spirit of the invention as set forth in the following claims.

1. A method for determining a patient's predisposition to develop coronary artery disease, comprising:
a. isolating DNA from a patient; and
b. analyzing the DNA to detect the presence of the KL-VS allele.

2. The method of claim 1 wherein detection of the KL-VS allele indicates the patient is predisposed to develop coronary artery disease.

3. A method of claim 1, wherein the detection of the KL-VS allele is characterized by diagnostic Mae III restriction fragments of 265 and 185 basepairs.

4. The method of claim 1, wherein detecting the KL-VS allele comprises RFLP analysis of a nucleic acid.

5. The method of claim 1, wherein detecting the KL-VS allele comprises amplification of a nucleic acid.

6. The method of claim 1, wherein the DNA is analyzed by:
a. amplifying the DNA in a polymerase chain reaction to produce an amplification product;
b. treating the amplified DNA with one or more restriction fragment enzymes; and
c. size fractionation of the amplification products.

7. The method of claim 6, wherein the polymerase chain reaction is performed with one or more oligonucleotides selected from the group consisting of:
sense primer 5′ AGGCTCATGCCAAAGTCTGG 3′ (SEQ ID No: 7); and
antisense primer 5′ GTTTCCATGATGAACTTTTTGAGG 3′ (SEQ ID No: 8).

8. The method of claim 7, wherein said one or more oligonucleotides are detectably labeled.

9. A method of predicting increased propensity for coronary artery disease in a patient, comprising:
detecting in a patient the presence of at least one copy of the KL-VS allele;
wherein detecting said allele indicates that said patient has an increased propensity for coronary artery disease.

10. A method for treating a patient suffering or susceptible to coronary artery disease, comprising:
selecting a patient that has a the KL-VS allele; and
treating the selected patient for coronary artery disease.

11. The method of claim 10 wherein the selected patient is treated by administering a therapeutic agent for coronary artery disease.

12. The method of claim 10 wherein the selected patient undergoes surgery for treatment.

13. The method of claim 10 wherein the selected patient undergoes a lifestyle change for treatment.

14. A diagnostic kit for coronary artery disease comprising means for analyzing DNA of a patient to detect the presence of the KL-VS allele.


SEQUENCE LISTING (6 sequences)

SEQ ID NO: 1 - protein, 549 aa, Homo sapiens [sequence omitted]

SEQ ID NO: 2 - protein, 1012 aa, Homo sapiens [sequence omitted]

SEQ ID NO: 3 - protein, 549 aa, Homo sapiens
MPASAPPRRPRPPPQSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDVTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVSQLTKPISSLTKPYH

SEQ ID NO: 4 - protein, 1012 aa, Homo sapiens
MPASAPPRRPRPPPQSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDVTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 5 - DNA, 20 bp, Artificial Sequence
  Description of Artificial Sequence Synthetic primer
aggctcatgccaaagtctgg

SEQ ID NO: 6 - DNA, 24 bp, Artificial Sequence
  Description of Artificial Sequence Synthetic primer
gtttccatgatgaactttttgagg